(19) United States
(12) Patent Application Publication
(10) Pub. No.: US 2016/0256460 A1
(43) Pub. Date: Sep. 8, 2016

(54) ACTIVATORS OR STIMULATORS OF SOLUBLE GUANYLATE CYCLASE FOR USE IN TREATING CHRONIC FATIGUE SYNDROME

(71) Applicant: BERGEN TEKNOLOGIOVERFØRING AS, Bergen (NO)

(72) Inventors: Öystein FLUGE, Morvik (NO); Olav MELLA, Olsvik (NO)

(21) Appl. No.: 15/032,831

(22) PCT Filed: Nov. 3, 2014
(86) PCT No.: PCT/EP2014/073538

§ 371 Date: Apr. 28, 2016

Related U.S. Application Data
(60) Provisional application No. 61/898,638, filed on Nov. 1, 2013.

(51) Int. Cl. A61K 31/506; A61K 39/395; A61K 31/197

(52) U.S. Cl. CPC A61K 31/506; A61K 31/197; A61K 39/3955; A61K 2039/505

(57) ABSTRACT

The present invention relates in a first aspect to a therapeutically effective amount of a stimulator of the soluble guanylate cyclase and/or of an activator of the soluble guanylate cyclase for use in the treatment of chronic fatigue syndrome (CFS) in a patient in need thereof. In a further aspect, the present invention relates to a combination of the stimulator and/or activator of the soluble guanylate cyclase with a B-cell depleting agent in the treatment of chronic fatigue syndrome. In addition, a combination of stimulator and/or activator of the soluble guanylate cyclase with B-Cell depleting agent is described. Said combination may be provided in form of a kit comprising suitably effective dosages of said compounds.

The present invention relates in a first aspect to a method for the treatment of chronic fatigue syndrome (CFS) comprising administering to a patient in need thereof a therapeutically effective amount of a stimulator of the soluble guanylate cyclase and/or a therapeutically effective amount of an activator of the soluble guanylate cyclase. In a further aspect, the present invention relates to a combination of the stimulator and/or activator of the soluble guanylate cyclase with a B-cell depleting agent in the treatment of chronic fatigue syndrome. In addition, a combination of stimulator and/or activator of the soluble guanylate cyclase and B-Cell depleting agent are described. Said combination may be provided in form of a kit comprising suitably effective dosages of said compounds.

### TECHNICAL BACKGROUND

Chronic fatigue syndrome (CFS) also described as myalgic encyophalitis (ME) is a disease affecting approximately 0.2 percent of the population (Nacul et al, BMC med 2011, 9:91). It is a disease affecting women three to four times more often than men and often preceded by an infection. It is speculated on a genetic predisposition for CFS (Albright et al, 2011, BMC neurol 11:62). According to the clinical working case definition (Canadian criteria) for CFS/ME, Carruthers B. M., et al., 2003, J. Chronic Fatigue Syndr, 11:7-36, the main symptoms are post-exertional malaise, with cognitive disturbances, pain, sensory hypersensitivity, and several symptoms related to neuroendocrine and autonomic function. CFS is characterized by an unexplained, severe fatigue, persisting for at least six consecutive months, and with a substantial reduction of previous levels in occupational, social or personal activities. Although many studies have been shown subtle alterations in blood tests or radiological investigations, no biomarker or diagnostic test exist.

That is, the aetiology of CFS remains unclear. Various hypotheses include immunological, virological, neuroendocrinological, and psychological mechanisms. The pathogenesis of CFS is presumed to be multifactorial and to involve both host and environmental factors.

Many patients suffering from CFS have a history of an acute viral infection preceding the development of fatigue. However, no persisting viral infection has been proven yet.

Furthermore, several gene expression studies have been performed in CFS, indicating that there are specific but complex gene alterations in accordance with the dysfunction in immune response and in defence mechanisms. For example, Kaushik N., et al., 2005, J. Clin Pathol 58:826-32 describe a microarray study showing differential expression of 16 genes in CFS suggesting T-cell activation and a disturbance of neuronal and mitochondrial function. Other microarray studies concluded that several genes affected mitochondrial function and cell cycle deregulation. Moreover, alterations in membrane transport and ion channels were described. Based on the numerous studies, the gene expression data are not conclusive but suggest that there are disturbances in CFS representing various cellular functions.

A study in 2007 of post-infective fatigue syndrome found no differences in ex vivo cytokine production over a 12-month period, as compared to controls recovering promptly after infection (Vollmer-Gonna U., et al., 2007, Clin Infect Dis 45:732-5). It is speculated that CFS patients may have a reduced immune cell function with a low NK cell cytotoxicity and immunoglobulin deficiencies.

For example, Ogava M., et al., 1998, Eur J of Clin Invest, 28:937-943 describes decreased nitric oxide mediated natural killer cell activation in chronic fatigue syndrome. It is identified therein, that NO donor were able to stimulate NK cell activity in healthy control subjects but not in NK cells obtained from CFS patients and stimulated in vitro.

Studies demonstrated several abnormalities in laboratory markers associated with immune functions in CFS patients. For example, a low NK cell cytotoxicity, but also an increase in CD8+ T cells, elevated numbers of CD20+ B-cells, and an increase in the B-cell subset expressing CD20 and CD5 has been described. A study comparing CFS patients and controls, reported decreased expression of CD69 on T cells in NK cells after mitogenic stimulation in vitro, indicating a disorder in the early activation of cellular immunity mediated by the cells (Mihaylova I., et al., 2007, Neuro Endocrinol Lett 28:477-83).

However, the data on immune bioregulation in CFS are not consistent, e.g. as discussed in Brenu et al, 2012, J Trans Med 10:88.

Along with hypotheses of immune deregulation in CFS, autoimmunity to endogenous vasoactive neuropeptides has been proposed as a mechanism for the disease, Staines D R., 2005, Med Hypotheses 64:539-42, however not supported by scientific data. Other reports are discussing various autoantibodies in conjunction with CFS. However, no clear association was proved. Thus, there is no direct evidence with consistent data for the presence of pathogenic autoantibodies or for T-lymphocyte mediated autoimmunity. That is, CFS is at present not defined as an autoimmune disease. Rather, CFS is still identified as a disease with unknown aetiology.

Various hypotheses for CFS pathogenesis are discussed in the art including blood platelet dysfunction, neurological, neuroendocrine, metabolic or autonomic disturbances, ion channel dysfunction, zinc deficiency, toxin exposure or prior vaccination, etc., However, no consistent picture has emerged for the aetiology and pathogenesis of CFS. Due to the lack of knowledge of the exact pathogenesis and with no known causal mechanism, there is no current standard specific treatment for CFS. The unknown aethiology of CFS is probably the reason for the remarkably few studies performed, evaluating therapy based upon a biological hypothesis.

Studies are described in the art testing treatment with immunoglobuline, or treatment with anti-viral compositions, like valganciclovir.

The inventors of the present invention published a Case series, Fluge O., Melia O., 2009, BMC Neurol. 9:28 followed by a double-blinded and placebo controlled, randomized phase II study, Fluge O., et al., 2011, PLOS 6:e26359, exploring B-cell depletion using the therapeutic monoclonal anti-CD20 antibody Rituximab, showing a clinical benefit in ⅔ of CFS patients. The use of B-cell depleting agents is described in WO 2009/083602. The patterns of responses and relapses, with a time delay of 2 to 8 eight months from start of Rituximab infusion (with rapid B-cell depletion) until start of clinical responses, indicate that an antibody may be involved in the pathogenesis. Recently, a case control study performed in elderly aged more than 65 years, investigating more than 1 million cases in cancer and 100,000 healthy controls, with a prevalence of CFS diagnosis of 0.5 percent in both groups, show that elderly CFS patients had a modest but highly significant risk of B-cell lymphomas, Chang C. M., Cancer, 2012, 118:5929-36, consistent with a chronic B-cell activation. Taken together, the data on treatment with a monoclonal anti-CD20 antibody examplified by Rituximab indicate that CFS in a subset of patients may be a post-infectious immune dysregulation, possibly a variant of autoimmune mechanisms, possibly with a genetic predisposition, in which B lymphocytes are important for symptom maintenance.

The guanylate cyclase, also known as guanylyl cyclase or guanyl cyclase, EC 4.6.1.2, (GC) is a lyase enzyme catalysing deformation of guanosine 3′,5′-monophosphate (cGMP) from guanosine triphosphate (GTP) and is found in tissues throughout the animal kingdom. Soluble GC (sGC) is the receptor for nitric oxide (NO) in vascular smooth muscle. In the cardiovascular system, NO is endogenously generated by endothelial NO synthase. Involvement of NO and sGC is described in stress and it appears that the NO-sGC-cGMP pathway is relevant for the control of a number of physiologic processes, including neuronal transmission, host defense, cell growth and proliferation as well as vascular and platelet homeostasis.

Stimulation of sGC mediates physiologic responses including smooth muscle relaxation, inhibition of inflammation and thrombosis. Hence, sGC represents a target for drugs in the treatment of various diseases. In general, two classes of compounds have been developed, that can directly activate sGC in pathophysiologic conditions when NO formation and bioavailability are impaired or when NO tolerance has developed. That is, heme-dependent stimulators and heme-independent activators of sGC have been described. For example, potent stimulators are described in a series of patent applications, e.g. WO 03/097063, WO 02/42302, WO 02/42299, WO 03/095451 and WO 03/004503.

Other activators of sGC are disclosed in WO 01/19780 or WO 01/19776. All documents including the compounds disclosed therein are included herein by reference. Furthermore, soluble guanylate cyclase activators are described in WO 2009/032249. A review of stimulators and activators of sGC and their potent therapeutic indications is provided in Nossaman B., et al., 2012, Critical Care Research and Practice, doi: 10.1155/2012/290805. Effectiveness of e.g. Cinaciguat, an activator of sGC is known. Cinaciguat also known as BAY 58-2667 is an experimental drug for the treatment of acute decompensated heart. In addition, Riociguat (BAY 63-2521), also known under the trade name Adempas, has been described as a stimulator of soluble guanylate cyclase. Clinical trials are underway to treat two forms of pulmonary hypertension, namely chronic tromboembolic pulmonary hypertension and pulmonary arterial hypertension.

However, as indicated above, using B-cell depletion by the monoclonal anti-CD20 antibody Rituximab in CFs, there is a significant delay from start of the treatment and the beginning of the symptoms relief. Also, the clinical studies performed with Rituximab, approximately ⅔ of CFS patients have a clinical response. This is not only true for treatment with Rituximab, but can also be seen with treatment of methotrexate, a small molecule known as an active agent for B-cell depletion useful in the treatment of various kinds of diseases.

Thus, there is an ongoing need to provide additional compounds useful in the treatment of chronic fatigue syndrome. In particular, there is an ongoing need for providing compounds which act fast in the patients without the lag period described for the B-cell depleting agent. In addition, there is a continuous demand for compounds which may also be effective in patients not susceptible to B-cell depleting agent treatment.

CFS patients have a marked endothelial dysfunction assessed by FlowMediated Dilation (FMD), a test that (under standardized conditions) largely reflect Nitric Oxide (NO) synthesis in endothelial cells after shear stress. A markedly reduced FMD, transient clinical responses after long-acting nitrates (like isosorbide mononitrate) and the clinical picture of CFS, are the basis for a hypothesis according to the present invention in which a main mechanism for CFS symptom maintenance is a relative lack of endothelial-cell derived Nitric Oxide (NO) availability. This results in reduced NO diffusion from endothelial cells to surrounding cells such as smooth muscle cells in blood vessel walls, and with a resulting inadequate regulation of blood flow to meet the metabolic demands of tissues. Also a relative lack of endothelial-cell derived NO may result in cognitive disturbances, sleep problems, a low anaerobic threshold, and lactate accumulation in tissues after modest exertion, a low NK cell function, all reported to be associated with CFS.

### DESCRIPTION OF THE PRESENT INVENTION

In a first aspect, the present invention relates to a method for the treatment of chronic fatigue syndrome (CFS) comprising administering to a patient in need thereof a therapeutically effective amount of a stimulator of the soluble guanylate cyclase (sGC) and/or a therapeutically effective amount of an activator of the soluble guanylate cyclase.

That is, the present inventors recognized that administration of an activator of sGC and/or a stimulator of sGC, e.g. Cinaciguat or Riociguat, relieve the symptoms of CFS and, thus, may be useful in the treatment of CFS, accordingly.

In particular, the present inventors recognized that an immediate relief, e.g. within a week, from start of administration of said activator or stimulator of sGC, e.g. by carefully increasing the dose, can be observed. In contrast to medication such as Rituximab for a treatment of CFS, which is characterized by a remarkable lag time before clinical responses, as described, e.g. WO 2009/083602. Hence, the administration of the stimulator and activator of sGC surprisingly allow a treatment of CFS patients for early relief of symptoms without a long delay as described for e.g. a B-Cell depleting agent, like Rituximab. It is has been recognized by the inventors that the sGC is involved in therapy of CFS patients.

In the context of the present invention, the terms “chronic fatigue syndrome”, CFS, and “Myalgic Encephalitis”, are used synonymously.

As used herein, the term “stimulator of the soluble guanylate cyclase” refers to compounds which stimulate sGC in a heme-dependent fashion. That is, the stimulators are particularly useful when in pathophysiological conditions NO formation and bioavailability are impaired or when NO tolerance has developed.

Further, the term “activators of soluble guanylate cyclase” refers to compounds acting heme independently on sGC. That is, the oxidation of the heme-iron on sGC decreases the responsiveness of enzyme to NO and promotes vasoconstriction. The activators of sGC target NO receptor protein when the heme-iron on sGC is in an oxidized state or when the heme-group is lost. The activators activate the oxidized or heme-deficient sGC enzyme that is not responsive to NO. Examples of the activator of sGC include the compound Cinaciguat. Examples of suitable stimulators of sGC include the compound Riociguat as described.

In an embodiment of the stimulator of the sGC the compound contains a pyrazol group preferably a pyrazolopyridine group.

For example, in an embodiment of the present invention the activator of the soluble guanylate cyclase is a compound having a structural element of general formula I

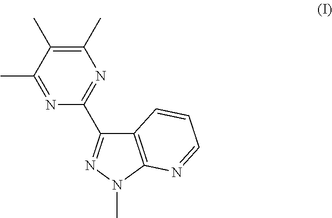

or a salt or hydrate thereof.

For example, the compound having the structure element of general formula I may be a compound of general formula II

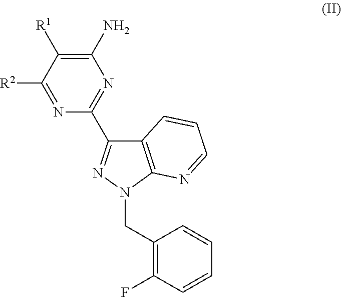

in which
R¹ is NR³C(═O)OR⁴, —O—SO₂—R⁴, phenyl or pyridine which may be substituted,

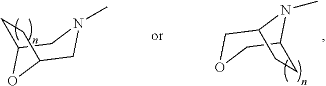

wherein n is 1 or 2,
R⁵NCOR⁶ wherein R⁵ and R⁶ together with the amide group to which they are bonded form a five- to seven-membered heterocycle which may be saturated or partially unsaturated, may optionally contain a further heteroatom chosen from N, O, and S, and may have 1 to 5 further substituents chosen from oxo, C1-6-alkyl, hydroxyl, hydroxy-C1-6-alkyl, and halogen, and may be fused to a C6-10-aryl ring or to a C3-8-cycloalkyl ring in which two carbon atoms are optionally connected together via an oxygen atom, or —NR⁷SO2R⁸ wherein R⁷ and R⁸ together with the heteroatoms to which they are bonded form a five- to seven-membered heterocycle which may be saturated or partially unsaturated, may optionally contain one or more other heteroatoms from the group of N, O, S, and may optionally be substituted;
R² is hydrogen or NH₂;
R³ is hydrogen or (C₁-C₄)-alkyl;
R⁴ is (C₁-C₆)-alkyl;
or a salt or hydrate thereof.
   For example, the stimulator of sGC is a compound of formula II

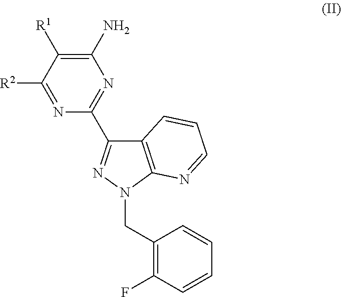

in which
R¹ is NR³C(═O)OR⁴,
R² is hydrogen or NH₂,
R³ is hydrogen or (C₁-C₄)-alkyl,
R⁴ is (C₁-C₆)-alkyl,
or a salt or hydrate thereof.
   Embodiments of the compound of the general formula III are embodiments wherein the stimulator of the soluble guanylate cyclase is a compound of the formula (II) in which
R¹ is NR³C(═O)OR⁴,
R² is hydrogen or NH₂,
R³ is (C₁-C₄)-alkyl,
R⁴ is (C₁-C₄)-alkyl,
or a salt or hydrate thereof,
   like wherein in the compound of the formula (II)
R¹ is —NR³C(═O)OR⁴,
R² is NH₂,
R³ is methyl or ethyl,
R⁴ is methyl, ethyl or isopropyl,
or a salt or hydrate thereof.
   In particular, the stimulator of the soluble guanylate cyclase is of the following structure:
Methyl 4,6-diamino-2-[1-(2-fluorobenzyl)-1H-pyrazolo[3,4-b]pyridine-3-yl]-5-pyrimidinyl(methyl)carbamate

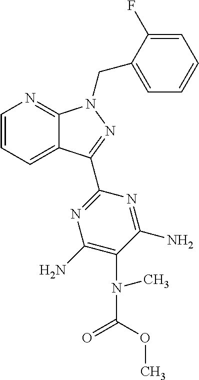 [SMILES: COC(=O)N(C)c1c(N)nc(-c2nn(Cc3ccccc3F)c3ncccc23)nc1N]

or a salt or hydrate thereof also known as BAY 63-2521 (Riociguat).
   Another suitable compound is a stimulator of the sGC of formula III

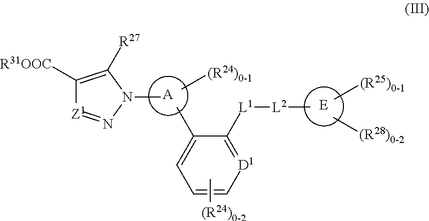

wherein
Z¹ is selected from the group consisting of CH and N;
A is a ring selected from the group consisting of

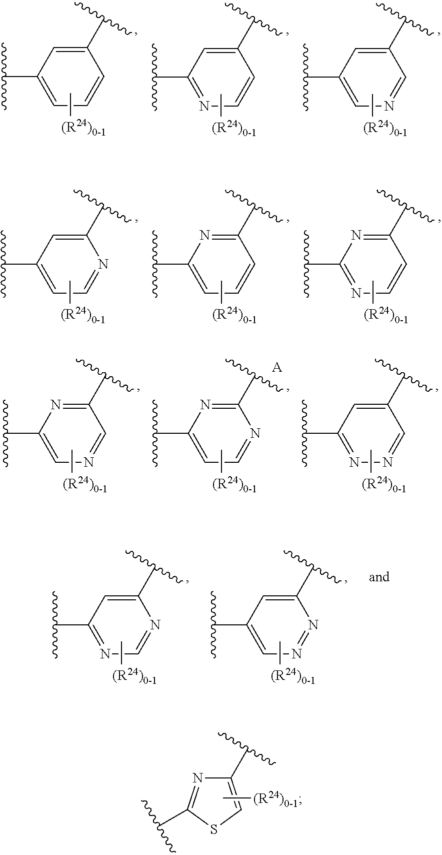

D¹ is CH, CR²⁴ or N;
R²⁷ is selected from the group consisting of
1) hydrogen,
2) C₁₋₆ alkyl wherein the alkyl group may be unsubstituted or substituted with 1-3 fluorine atoms and unsubstituted or monosubstituted with OC₁₋₃ alkyl,
3) C₃₋₆ cycloalkyl wherein the cycloalkyl group may be unsubstituted or substituted with 1-3 fluorine atoms and unsubstituted or monosubstituted with OC₁₋₃ alkyl, and
4) phenyl, wherein the phenyl group is unsubstituted or substituted with C₁₋₄ alkyl, —OC₁₋₄ alkyl, halogen, CN, NO₂, and S(O)₀₋₂C₁₋₄ alkyl, wherein C₁₋₄ alkyl and —OC₁₋₄ alkyl are unsubstituted or substituted with 1-3 flourine atoms;
L¹ is selected from the group consisting of O, S, C(R³²)2; and CF₂;
L² is selected from the group consisting of (CH₂)₂₋₄, —C(R³²)₂, —CF₂—O, and
S, provided that when L¹ is O or S, L² is not O or S;
R³² is independently selected from the group consisting of hydrogen and C₁₋₃ alkyl, wherein C₁₋₃ alkyl is unsubstituted or substituted with 1-3 flourine atoms;
E is a ring selected from the group consisting of
1) a 6-10 membered aryl ring
2) a 5-10 membered heteroaryl ring having 1, 2 or 3 heteroatoms independently selected from the group consisting of 0, 1, 2 and 3 N atoms, 0 or 1 O atoms, and 0 or 1 S atoms,
3) a C₃₋₈ cycloalkyl ring; wherein aryl, heteroaryl, and C₃₋₈ cycloalkyl are unsubstituted or monosubstituted with R²⁵, and unsubstituted or monosubstituted with R²⁵, and unsubstituted, monosubstituted or independently disubstituted with R²⁸,
R²⁴ in each instance in which it occurs, is independently selected from the group consisting of halogen,
C₁₋₆ alkyl, wherein the alkyl group may be unsubstituted or substituted with 1-3 fluorine atoms,
—O—C₁₋₆ alkyl, wherein the alkyl group may be unsubstituted or substituted with 1-3 fluorine atoms,
C₃₋₈ cycloalkyl, unsubstituted or substituted with 1-3 fluorine atoms, CN, and
NO₂;
R²⁵, in each instance in which it occurs, is independently selected from the group consisting of
1) R²⁶,
2) —OR²⁶,
3) C₁₋₆ alkyl which may be unsubstituted or substituted with 1-3 fluorine atoms, and unsubstituted or monosubstituted with a group independently selected from C₃₋₆ cycloalkyl, —O—C₁₋₄ alkyl, OH, ═O, S(O)₀₋₂C₁₋₄ alkyl, —OR²⁶ and R²⁶,
4) C₁₋₆ alkenyl which may be unsubstituted or substituted with 1-3 fluorine atoms and unsubstituted or monosubstituted with a group independently selected from —O—C₁₋₄ alkyl, OH, —O, S(O)₀₋₂C₁₋₄ alkyl, —OR²⁶ and R²⁶,
5) O—C₁₋₆ alkyl wherein the alkyl group may be unsubstituted or substituted with 1-3 fluorine atoms, and unsubstituted or monosubstituted with a group independently selected from C₃₋₆ cycloalkyl and R²⁶,
6) —S—C₁₋₆ alkyl,
7) a C₃₋₈ cycloalkyl ring which is unsubstituted or mono, di- or trisubstituted with groups independently selected from fluoro and C₁₋₄ alkyl, and unsubstituted or monosubstituted with a group independently selected from C₁₋₄ alkyl wherein the alkyl group may be unsubstituted or substituted with 1-3 fluorine atoms, —O—C₁₋₄ alkyl, OH, ═O, S(C)₀₋₂C₁₋₄ alkyl, —OR²⁶, R²⁶, and NR²⁹R³⁰,
8) a C₅₋₈ cycloalkenyl ring which is unsubstituted or mono, di- or trisubstituted with a group independently selected from fluoro and C₁₋₄ alkyl, and unsubstituted or monosubstituted with a group independently selected from C₁₋₄ alkyl, wherein the alkyl group may be unsubstituted or substituted with 1-3 fluorine atoms, —O—C₁₋₄ alkyl, OH, ═O, S(O)₀₋₂C₁₋₄ alkyl, and R²⁶,
9) a 5- to 6 membered heterocyclyl ring having 1 or 2 heteroatoms selected from the group consisting of N, O and S, and which is unsubstituted or monosubstituted with a group independently selected from C₁₋₄ alkyl wherein the alkyl group may be unsubstituted or substituted with 1-3 fluorine atoms, —OC₁₋₄ alkyl and ═O, and 10) halogen;
R²⁶ is selected from the group consisting of
1) a phenyl ring which is unsubstituted, monosubstituted or disubstituted with a group independently selected from the group consisting of halogen, OH, CN, C₁₋₄ alkyl wherein the alkyl group may be unsubstituted or substituted with 1-3 fluorine atoms, OC₁₋₄ alkyl wherein the alkyl group may be unsubstituted or substituted with 1-3 fluorine atoms, NO₂, S(O)₀₋₂C₁₋₄ alkyl, C₂₋₄ alkenyl, O—C₂₋₄ alkenyl, NR²⁹R³⁰, and COOH, and
2) a 5-6 membered heteroaryl ring containing 1-2 heteroatoms which are independently selected from N, O and S, wherein the heteroaryl ring is unsubstituted, monosubstituted or disubstituted with a group independently selected from: halogen, OH, CN, C₁₋₄ alkyl wherein the alkyl group may be unsubstituted or substituted with 1-3 fluorine atoms, OC₁₋₄ alkyl wherein the alkyl group may be unsubstituted or substituted with 1-3 fluorine atoms, NO₂, S(O)₀₋₂C₁₋₆ alkyl, S(O)₀₋₂ aryl, C₂₋₆ alkenyl, OC₂₋₆ alkenyl, NR²⁹R³⁰, and COOH;
R²⁸ is selected from the group consisting of
C₁₋₄ alkyl wherein the alkyl group may be unsubstituted or substituted with 1-3 fluorine atoms,
C₂₋₄ alkenyl,
halogen,
C₃₋₆ cycloalkyl, wherein the cycloalkyl group may be unsubstituted or substituted with 1-3 fluorine atoms,
OC₁₋₄ alkyl wherein the alkyl group may be unsubstituted or substituted with 1-3 fluorine atoms,
O—C₂₋₄ alkenyl,
NO₂,
S(O)₀₋₂C₁₋₄ alkyl, and
CN;
R²⁹ and R³⁰ are independently selected from the group consisting of hydrogen and C₁₋₆alkyl;
and
R³¹ is selected from the group consisting of hydrogen and C₁₋₆ alkyl. or a pharmaceutically acceptable salt or hydrate thereof.

Furthermore, suitable compounds are activators of the sGC, e.g. activators having the structure of general formula IV

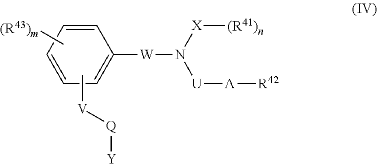

in which V is absent, O, S, or NR″, in which R⁴⁴ is hydrogen or methyl,
Q is absent, straight-chain or branched alkylene having up to 9 carbon atoms or straight-chain or branched alkenediyl or straight-chain or branched alkinediyl having up to 4 carbon atoms which may be monosubstituted by halogen,
Y is H, NR⁴⁸R⁴⁹, cyclohexyl, phenyl, naphthyl or a heterocycle from the group consisting of

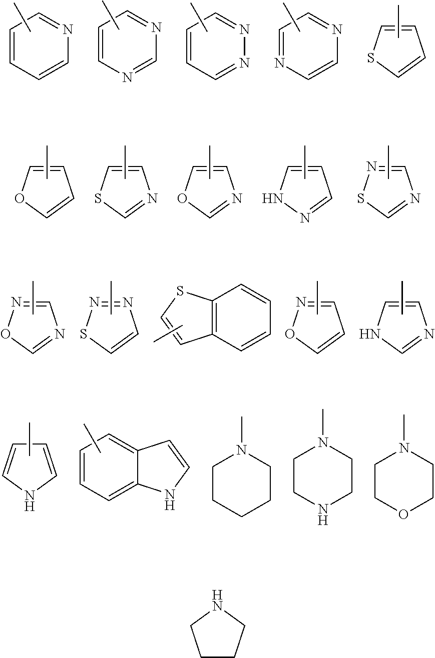

which may also be attached via N,
where the cyclic radicals may in each case be mono-, di or trisubstituted by straight-chain or branched alkyl, straight-chain or branched alkenyl, straight-chain or branched alkinyl, straight-chain or branched alkoxy, straight-chain or branched alkoxyalkoxy, straight-chain or branched halogenalkyl, straight-chain or branched halogenoalkoxy having in each case up to 4 carbon atoms, straight-chain or branched cycloalkyl having 3 to 6 carbon atoms, F, Cl, Br, I, NO₂, SR⁴⁶, NR⁴⁸R⁴⁹, NR⁴⁷COR⁵⁰, or CONR⁵¹R⁵², in which
R⁴⁶ is hydrogen, straight-chain or branched alkyl having up to 8 carbon atoms, or straight-chain or branched halogenoalkyl having up to 4 carbon atoms,
R⁴⁷ is hydrogen, or straight-chain or branched alkyl having up to 4 carbon atoms, R⁴⁸, R⁴⁹, R⁵¹ and R⁵² independently of one another are hydrogen, straight-chain or branched alkyl having up to 4 carbon atoms or phenyl, where the phenyl radical may be mono-, di- or trisubstituted by F, Cl, Br, hydroxyl, methyl, ethyl, n-propyl, i-propyl, n-butyl, s-butyl, i-butyl, t-butyl, methoxy, ethoxy, amino, acetylamino, NO₂, CF₃ OCF₃ or CN, or two substituents R⁴⁸ and R⁴⁹ or R⁵¹ and R⁵² may be attached to one another forming a five- or six-membered ring which may be interrupted by O or N,
R⁵⁰ is hydrogen, straight-chain or branched alkyl having up to 4 carbon atoms or phenyl,
   where the phenyl radical may be mono- to trisubstituted by F, Cl, Br, hydroxyl, methyl, ethyl, n-propyl, i-propyl, n-butyl, s-butyl, i-butyl, t-butyl, methoxy, ethoxy, amino, acetylamino, NO₂, CF₃, OCF₃ or CN;
      and/or the cyclic radicals may in each case be mono-, di- or trisubstituted by phenyl or a heterocycle from the group consisting of

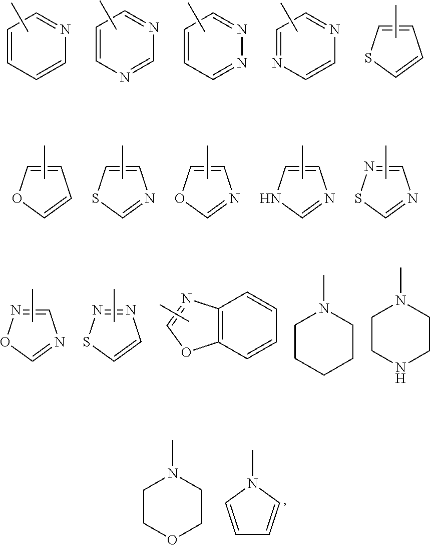

which may be attached directly or via a group O, S, SO, SO₂, NR⁴⁴, SO₂NR⁴⁷, CONR⁴⁷, straight-chain or branched alkylene, straight-chain or branched alkenediyl, straight-chain or branched alkyloxy, straight-chain or branched oxyalkyloxy, straight-chain or branched sulphonylalkyl, straight-chain or branched thioalkyl having in each case 4 carbon atoms and which may be mono- to trisubstituted by straight-chain or branched alkyl, straight-chain or branched alkoxy, straight-chain or branched alkoxyalkoxy, straight-chain or branched halogenoalkyl or straight-chain or branched alkenyl having in each case up to 4 carbon atoms, F, Cl, Br, I, CN, SCHL₃, OCF₃, NO₂, NR⁴⁸R⁴⁹ or NR⁵⁴COR⁵⁷,
in which
R⁵⁴ is hydrogen, straight-chain or branched alkyl having up to 8 carbon atoms, or cycloalkyl having 3 to 8 carbon atoms, and
R⁵⁷ is hydrogen, straight-chain or branched alkyl having up to 12 carbon atoms, straight-chain or branched alkenyl having up to 12 carbon atoms, aryl having 6 to 10 carbon atoms, an aromatic heterocycle having 1 to 9 carbon atoms and up to 3 heteroatoms from the group consisting of S, N and O or cycoalkyl having 3 to 8 carbon atoms, which may furthermore optionally be substituted by F, Cl Br, hydroxyl, methyl, ethyl, n-propyl, i-propyl, n-butyl, s-butyl, i-butyl, t-butyl, methoxy, ethoxy, amino, acetylamino, CO₂, CF₃, OCF₃ or CN;
   and/or the cyclic radicals may be fused with an aromatic or saturated carbocycle having 1 to 10 carbon atoms or an aromatic or saturated heterocycle having 1 to 9 carbon atoms and up to 3 heteroatoms from the group consisting S, N and O,
R⁴³ is hydrogen or fluorine,
m is an integer from 1 to 2,
W is CH₂, —CH₂CH₂—, CH₂CH₂CH₂, CH═CHCH₂,
U is —CH₂—,
A is phenyl, pyridyl, thienyl or thiazolyl which may optionally be mono- to trisubstituted by methyl, ethyl, n-propyl, i-propyl, n-butyl, i-butyl, s-butyl, t-butyl, CF₃, methoxy, ethoxy, F, Cl, Br,
R⁴² is COOR⁶⁴, in which
   R⁶⁴ is hydrogen or straight-chain or branched alkyl having up to 4 carbon atoms,
X is straight-chain or branched alkylene having up to 8 carbon atoms or straight-chain or branched alkenediyl having up to 8 carbon atoms which may in each case contain one to three groups from the group consisting of phenyl, phenyloxy, O, CO and CONR⁷⁰,
   in which
   R⁷⁰ is hydrogen, straight-chain or branched alkyl having up to 6 carbon atoms or cycloalkyl having 3 to 6 carbon atoms,
n is 1 or 2,
R⁴¹ is COOR⁷⁵, in which
   R⁷⁵ is hydrogen or straight-chain or branched alkyl having up to 6 carbon atoms.
      For example, the activator is a compound of the following structure

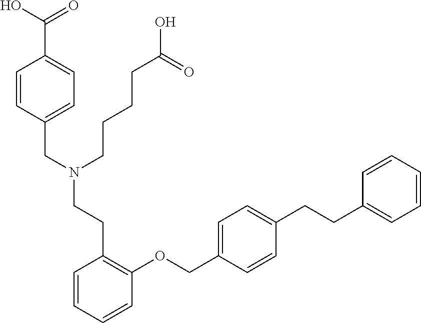 [SMILES: O=C(O)CCCCN(CCc1ccccc1OCc1ccc(CCc2ccccc2)cc1)Cc1ccc(C(=O)O)cc1]

representing Cinaciguat, also known as BAY 58-2667.

An embodiment of the present invention, a combination of at least one stimulator of sGC and at least one activator of sGC may be provided.

The route of administration of the stimulator and/or activator of sGC depends on the formulation used. That is, the stimulator and/or activator may be administered in form of capsules or other suitable forms, like tablets.

In addition, the activator and/or stimulator may be in a form of a compound of immediate relief or in a form of a delayed or sustained thereof. Furthermore, if applicable, the activator and/or stimulator may be provide in powder formed for oral use.

For example, the activator and/or stimulator is adapted for systemic administration, for example via the enteral or parenteral route. In another embodiment, the activator and/or stimulators is adapted for mucosal or local administration.

Moreover, in another embodiment, the activator and/or stimulator useful in the treatment of CFS is adapted for the administration to a subject in a single therapeutically effectiv dosis or multiple of therapeutically effective dosis thereof. The skilled person is well aware of the effective dose to be administered. Typically, the daily doses is similar to the daily doses administered in the treatment of other diseases treated with said stimulators and/or activators of sGC. Clinical trials with Riociguat for pulmonary hypertension used oral doses up to 2.5 mg three times daily (Ghofrani et al, NEJM, 369:4, 2013). Clinical trials with Cinaciguat for acute heart failure used intravenous infusions, in doses starting at 100 microg/hour (Erdmann et al, Eur J Heart, 34:1, 2013).

Typically, the activator and/or stimulator of sGC useful in the treatment of CFS is in a suitable pharmaceutical form, for example, in combination with a pharmaceutically acceptable diluent, excipient or carrier. The pharmaceutical composition may contain additional components including pharmaceutical additives, pH-stabulizer, etc.

That is, the present invention provides a pharmaceutical composition comprising the activator and/or stimulator of sGC as defined herein and a pharmaceutically acceptable diluent, excipient or carrier useful in the treatment of CFS.

The dosis and administration is similar to the dosis and administration as described for the activator and/or stimulator sGC in connection with other diseases and disorders. That is, for example for Adempas containing the active ingredient riociguate, the dosis and administration is initially 0.5 to 2 mg daily gradually increasing the same over the first days. For example, after five days the dosis is between 0.5 mg and 5 mg. The dosis may be increased by 0.5 mg or 1 mg at intervals to a daily dosage of 2 to 10 mg. The skilled artisan is well aware of suitable dosage. The daily dosage may be taken once daily or several times daily, e.g. two times daily or three times daily. That is, the initial daily dosage is in the range of 0.5 to 3 mg active ingredient administered once or three times daily while increasing the same over the treatment period. The typical maximum dosage is about 10 mg daily dosage, e.g. 2.5 mg three times a day.

That is, dosis and administration are similar to dosis and administration of the compounds described for other diseases, disorders or conditions.

The administration is e.g. in form of tablets having a dosage of between 0.5 to 2.5 mg per tablet. Of course, the compound may be administered by other ways, typically, administered systemically by known means. For example, for the active ingredient riociguate, the commercial products under the trade name Adempas of film tablets of 0.5 mg, 1 mg, 1.5 mg, 2 mg, and 2.5 mg may be administered according to the manufacturer's instructions.

As used herein, the term “comprising”, “comprises”, “containing” or “contains” includes the embodiments of “consiting of” or “consist”.

In another embodiment of the present invention relates to a composition containing a combination of an activator and/or stimulator of sGC as defined herein and a B-cell depleting agent. Said composition is particularly useful as a pharmaceutical composition, e.g. for use in the treatment of chronic fatigue syndrome.

That is, it is preferred that the pharmaceutical composition is a composition containing a combination of an activator and/or stimulator of sGC and a B-cell depleting agent for use in the treatment of chronic fatigue syndrome wherein the combination is administered simultaneously, separately or sequentially.

In a preferred embodiment, the pharmaceutical composition is designed to allow administration of the activator and/or stimulator of sGC in a pharmaceutically effective dosage over a time range of the first six weeks of treatment, preferably over a time range of the first eight weeks of treatment, like within three month or four month from the beginning of the treatment. Of course, the treatment regimen depends on the drug administered as well as on the way of administration. The skilled artisan is well aware of suitable dosages and treatment regimen depending on the drug. For example, the same dosages of the activator and/or stimulator of sGC drugs may be administered as it is the case for other types of diseases or disorders the same activator and/or stimulator of sGC are useful.

In addition, the pharmaceutical composition is designed that the B-cell depleting agent is adapted for administration 1 or 2 in fusions twice within the first two weeks and, there after, administering the B-cell depleting agent once every two or three month for maintaining the beneficial effect.

As used herein, the term “B-cell depletion” or “B-cell depleting activity” refers to the ability of the entity, either a chemical or biological entity, e.g. an antibody, to reduce circulating B-cell levels in a subject. B-cell depletion may be achieved e.g. by inducing cell death or reducing proliferation.

The “CD20” antigen, or “CD20,” is an about 35-kDa, non-glycosylated phosphoprotein found on the surface of greater than 90% of B cells from peripheral blood or lymphoid organs in humans. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sd. (USA) 82:1766 (1985), for example. The term CD20 includes the equivalent molecules of other species than human. Recently, low level expression of CD20 on a subset of T-cells and NK-cells has been reported.

A “B-cell” is a lymphocyte that matures within the bone marrow, and includes a naive B cell, memory B cell, or effector B cell (plasma cells).

In a broader sense, the present invention relates not only to the use of antibodies or fragments thereof for the treatment of CFS but to the use of antagonists of the CD20 molecule in general having a B-cell depleting activity for the treatment of CFS.

An “antagonist” or “B-cell depleting agent” which is used herein interchangeably is a molecule which, e. g. upon binding to a B cell surface marker, like CD20 on B cells, destroys or depletes B cells in a mammal and/or interferes with one or more B cell functions, e.g. by reducing or preventing a humoral response elicited by the B cell. The antagonist or B-cell depleting agent according to the present invention is able to deplete B cells (i.e. reduce circulating B cell levels) in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native sequence peptides and small molecule antagonists which bind to the B cell surface marker, optionally conjugated with or fused to a cytotoxic agent. A preferred antagonist is a CD20 antibody or CD20-binding antibody fragment. Furthermore, small molecule antagonists are preferred, like the known B-cell depleting agent Methotrexat.

Insofar that other cells than B-cells express the CD20 antigen like a subset of T-cells or NK-cells, these cells are also depleted with the B-cells depleting agent being an agent acting via CD20.

Antagonists which “induce apoptosis” are those which induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

The term “antibody” herein is used in the broadest sense and specifically covers monoclonal antibodies, polyclonal antibodies, multispecific antibodies {e.g. bispecific antibodies) formed from at least two intact antibodies, and antibody fragments so long as they exhibit the desired biological activity.

In a preferred embodiment, the antibody useful for the treatment of CFS is a B-cell depleting CD20-binding antibody fragment.

“CD20-binding antibody fragments” comprise a portion of an intact antibody which comprises the antigen binding region thereof. Examples of antibody fragments include Fab, Fab′, F(ab′)2, and Fv fragments; diabodies; linear antibodies; single-chain antibody molecules; and multispecific antibodies formed from antibody fragments. For the purposes herein, an “intact antibody” is one comprising heavy and light variable domains as well as an Fc region.

Moreover, it is assumed that other B-cell depleting agents, in particular, anti-CD22 antibodies, like Epratuzumab or anti-CD19 humanized antibodies, like MDX-1342, can be used for the treatment of CFS.

The terms “rituximab” or “RITUXAN®” or “mabthera” herein refer to the genetically engineered chimeric murine/human monoclonal antibody directed against the CD20 antigen and designated “C2B8” in U.S. Pat. No. 5,736,137, expressly incorporated herein by reference, including fragments thereof which retain the ability to bind CD20. Purely for the purposes herein and unless indicated otherwise, “humanized 2H7” refers to a humanized antibody that binds human CD20, or an antigen-binding fragment thereof, wherein the antibody is effective to deplete primate B cells in vivo.

The expression “effective amount” of the B-cell depleting agent or antagonist, in particular of the anti-CD20 antibody or CD20-binding antibody fragment thereof, refers to an amount of the B-cell depleting agent or antagonist which is effective for treating CFS. For example, the anti-CD20 antibody for the treatment of chronic fatigue syndrome/myalgic encephalomyelitis is administered in the range of 10 mg to 5000 mg per dosage. For example, the dosage may be in the range of from 100 to 1000 mg/m2, in particular, 500 mg/m2 as a single infusion for Rituximab. Typically, the dosage for Methotrexate is in the range of 5 mg to 30 mg per week.

In one preferred embodiment, the B-cell depleting agent is a chemical entity, e.g. a small molecule. A variety of B-cell depleting agents are known in the art for example known B-cell depleting agents are BAFF-antagonists. Furthermore, known B-cell depleting agents include antagonists of BR3, agonists of alpha-4-integrins etc. For example, Methotrexate is an analogue of folic acid displaying B-cell depleting activity. Other useful B-cell depleting agents are small modular immunopharmaceuticals (SMIP) against CD20. For example, SMIP acting as B-cell depleting agents are TRU-015 or SBI-087 of Trubion Pharmaceuticals. Also, SMIP can be single chain polypeptides, smaller than antibodies, having a potent B-cell depletion activity.

In a preferred embodiment, a combination of an anti CD20 antibody and representing a biological entity of a B-cell depleting agent and Methotrexat, representing a chemical entity of a B-cell depleting agent, are used for treating chronic fatigue syndrome of myalgic encephalomyelitis. Administration of these entities may be effected simultaneously, separately or sequentially. For example, in a first regimen either the antibody or Methotrexat is administered to the subject while in a second regimen the other agent is administered.

The composition comprising the B-cell depleting agent, the antagonist, in particular, the anti CD20 antibody or the CD20-binding antibody fragment thereof, will be formulated, dosed, and administered in a fashion consistent with good medical practice. Factors for consideration in this context include the stage of the particular disease or disorder being treated, the particular mammal being treated, the clinical condition of the individual subject, the site of delivery of the agent, the method of administration, the scheduling of administration, and other factors known to medical practitioners. The effective amount of the B-cell depleting agent, like an antibody or antibody fragment to be administered will be governed by such considerations. As a general proposition, the effective amount of the antagonist administered parenterally per dose will be in the range of about 20 mg/m² to about 10,000 mg/m² of subject body, by one or more dosages. Exemplary dosage regimens for intact antibodies include 375 mg/m2 weekly×4; 1000 mg×2 (e.g. on days 1 and 15); or 1 gram×3. The antibody for the administration to a subject in a single therapeutically effective dosage of said antibody is of 50 to 2000 mg/m² or multiple of therapeutically effective dosages of said antibody or CD20-binding antibody fragment thereof of 50 to 2000 mg/m2. As noted above, however, these suggested amounts of antibody are subject to a great deal of therapeutic discretion. The key factor in selecting an appropriate dose and scheduling is the result obtained, as indicated above. The B-cell depleting agent antagonist, like the antibody, is administered by any suitable means, including parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the B-cell depleting agent antagonist, like the antibody may suitably be administered by pulse infusion, e.g., with declining doses of the antagonist. Preferably the dosing is given by intravenous injections.

In another embodiment, the combination is a combination of a activator and/or stimulator of sGC and a B-cell depleting agent wherein the said B-cell depleting agent is Methotrexate.

Furthermore, a combination of an activator and/or stimulator of sGC and a nitric oxide donor is possible. The combination may be a mixture of both components or each component may be present separately. The components of the combination according to the present invention may be administered simultaneously, separately or sequentially.

Moreover, another embodiment of the present invention relates to a combination of at least three active agents, namely a combination an activator and/or stimulator of sGC as defined herein, a NO donor component and a B-Cell depleting agent.

Furthermore, the combination may be in form of at least two different components whereby each component may be separately administered. For example, while one component of the combination according to the present invention may be provided systemically, at least one other component may be adapted for local administration or mucosal administration.

### Pharmaceutical Formulations

Therapeutic formulations of the NO donor and, optionally, the B-cell depleting agents, like antibodies or other antagonists used in accordance with the present invention are prepared for storage by mixing an antibody or a fragment thereof having the desired degree of purity with optional pharmaceutically acceptable carriers, excipients or stabilizers {Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl parabene; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, PLURONICS™ or polyethylene glycol (PEG).

Exemplary anti-CD20 antibody formulations which may form the bases of the compositions according to the present invention are described in WO98/56418, expressly incorporated herein by reference. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5. Lyophilized formulations adapted for subcutaneous administration are described in U.S. Pat. No. 6,267,958 (Andya et ah). Such lyophilized formulations may be reconstituted with a suitable diluent to a high protein concentration and the reconstituted formulation may be administered subcutaneously to the mammal to be treated herein. Crystalized forms of the antibody or antagonist are also contemplated. See, for example, US 2002/0136719A1.

The active ingredients may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980). Sustained-release preparations may be prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethylmethacrylate), or poly(vinylalcohol)), polylactides (U.S. Pat. No. 3,773,919), nondegradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydiOxybutyric acid. The formulations to be used for in vivo administration must be sterile. This is readily accomplished by filtration through sterile filtration membranes.

Moreover, the present invention relates to a kit comprising

a) a first component which comprises an activator and/or stimulator of sGC as defined herein, and
b) a second component which comprises a B-cell depleting agent as defined herein.

It is preferred, that this kit is for use in the treatment of CFS.

It is preferred that the method for treating CFS with the an activator and/or stimulator of sGC comprises the administration of a suboptimal or low dosage at the beginning. In particular, the starting dosage may be reduced to avoid any undesired side effects. Over the time, the dosage may be increased to dosages as administered e.g. in angina pectoris.

Furthermore, the dosage may be an immediate release or sustained dosage depending on the way of administration.

The administration may be systemically or locally via the enteral or parenteral way. For example, topical administration may be affected, e.g. by a plaster for sustained release alternatively, an activator and/or stimulator of sGC may be administered by way of inhalation or by other mucosal ways.

In a preferred embodiment, the method of treatment comprises administration of the an activator and/or stimulator of sGC in combination with a B-cell depleting agent. That is, a combined treatment with an activator and/or stimulator of sGC and the B-cell depleting agent as defined herein is particularly preferred.

The both components may be administered simultaneously, separately or sequentially to said subject suffering from CFS. For example, an activator and/or stimulator of sGC may be administered by inhalation while the B-cell depleting agent is administered by the way of infusion. Furthermore, while an activator and/or stimulator of sGC may be administered on a daily basis, the B-cell depleting agent may be administered initially in once a week over two weeks and, thereafter, in a free determined time of schedule, e.g. every second or every third month.

In particular, an activator and/or stimulator of sGC may be administered during the initial 4 to 12 weeks, like during the first 4 to 8 weeks of treatment, e.g. the initial 4, 5, 6, 7, 8, 9, 10, 11, 12 weeks to allow immediate relief of the symptoms of CFS while the administration at the B-cell depleting agent as defined herein allows relief to the symptoms of the subject suffering from CFS over a long time period due to the delayed effectiveness thereof.

### Examples

The invention will be described further by a way of examples using riociguate (Adempas) demonstrating the effectiveness in the treatment of CFS. It is understood that the examples illustrate the invention further without limiting the same.

The patient is a 46 years old woman. She has had ME/CFS for 10 years, starting after a Giardia Lamblia infection. She has a disease severity varying between moderate and mild, but with moderate severity the last month prior to start of Riociguat. She has a typical ME/CFS and fulfils Canadian criteria. She has not been given Rituximab (anti-CD20 antibody) previously.

She started with only 0.5 mg once daily, gradually increasing to 0.5 two times daily after a few days and again to 0.5 mg three times daily on day six. Starting from day six, she noticed the next three days a benefit on the ME/CFS symptoms. The dose was increased to 1 mg+0.5 mg+1 mg starting from day seven and, in addition, starting from day nine increased to 1 mg three times daily. The patient has had no clear side effects.

After the first six days, at a dose of 2.5 mg per day, the patient started to feel more energy and less fatigue, less brain fog with an improved concentration ability, less hypersensitivity to sound and light, and less feeling of sore throat. She told the clinicians of the improvement of her ME/CFS symptoms. She experienced this improvement to be significant and not within the usual range of symptom variation of her ME/CFS disease the last months.

1. A method for the treatment of chronic fatigue syndrome (CFS) comprising administering to a patient in need thereof a therapeutically effective amount of a stimulator of the soluble guanylate cyclase or a therapeutically effective amount of an activator of the soluble guanylate cyclase.

2. The method according to claim 1 wherein said stimulator of the soluble guanylate cyclase or the activator of the soluble guanylate cyclase is a compound containing a pyrazole group preferably a pyrazolopyridine group.

3. The method according to claim 1 wherein the stimulator of the soluble guanylate cyclase or the activator of the soluble guanylate cyclase is a compound having the structural element of general formula (I): 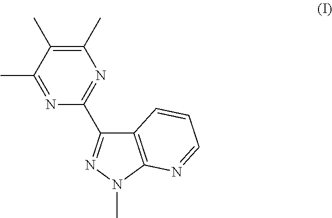
or a salt or hydrate thereof.

4. The method according to claim 3, wherein the stimulator of the soluble guanylate cyclase is a compound of formula (II): 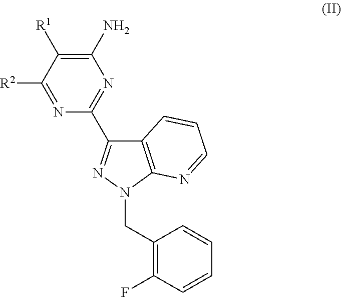
in which
R¹ is NR³C(═O)OR⁴, —O—SO₂—R⁴, phenyl or pyridine which may be substituted, 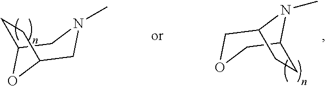
wherein n is 1 or 2,
R⁵NCOR⁶ wherein R⁵ and R⁶ together with the amide group to which they are bonded form a five- to seven-membered heterocycle which may be saturated or partially unsaturated, may optionally contain a further heteroatom chosen from N, O, and S, and may have 1 to 5 further substituents chosen from oxo, C1-6-alkyl, hydroxyl, hydroxy-C1-6-alkyl, and halogen, and may be fused to a C6-10-aryl ring or to a C3-8-cycloalkyl ring in which two carbon atoms are optionally connected together via an oxygen atom, or —NR⁷SO2R⁸ wherein R⁷ and R⁸ together with the heteroatoms to which they are bonded form a five- to seven-membered heterocycle which may be saturated or partially unsaturated, may optionally contain one or more other heteroatoms from the group of N, O, S, and may optionally be substituted;
R² is hydrogen or NH₂;
R³ is hydrogen or (C₁-C₄)-alkyl;
R⁴ is (C₁-C₆)-alkyl;
or a salt or hydrate thereof.

5. The method according to claim 3 wherein the stimulator of the soluble guanylate cyclase is a compound of formula (II) 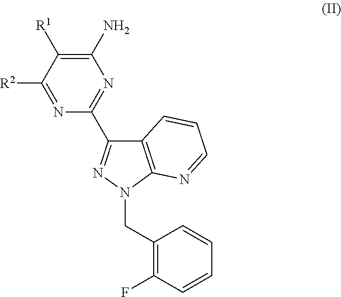
in which
R¹ is NR³C(═O)OR⁴,
R² is hydrogen or NH₂,
R³ is hydrogen or (C₁-C₄)-alkyl,
R⁴ is (C₁-C₆)-alkyl,
or a salt or hydrate thereof.

6. The method according to claim 5 wherein the stimulator of the soluble guanylate cyclase is a compound of the formula (II) in which
R¹ is NR³C(═O)OR⁴,
R² is hydrogen or NH₂,
R³ is (C₁-C₄)-alkyl,
R⁴ is (C₁-C₄)-alkyl,
or a salt or hydrate thereof.

7. The method according to claim 6 wherein in the compound of the formula (III)
R¹ is —NR³C(═O)OR⁴,
R² is NH₂,
R³ is methyl or ethyl,
R⁴ is methyl, ethyl or isopropyl,
or a salt or hydrate thereof.

8. The method according to claim 5, wherein the stimulator of the soluble guanylate cyclase is the following structure:
Methyl 4,6-diamino-2-[1-(2-fluorobenzyl)-1H-pyrazolo[3,4-b]pyridine3-yl]-5-pyrimidinyl(methyl)carbamate 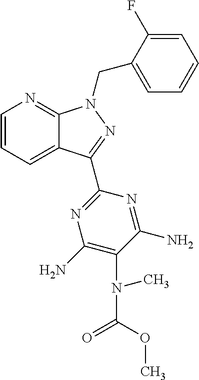 [SMILES: COC(=O)N(C)c1c(N)nc(-c2nn(Cc3ccccc3F)c3ncccc23)nc1N]
or a salt or hydrate thereof.

9. The method according to claim 1 wherein the stimulator of the soluble guanylate cyclase is a compound of formula (III) 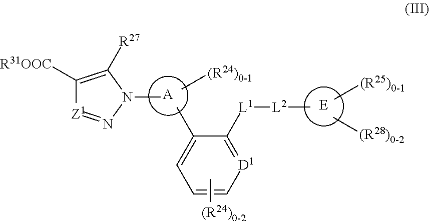
wherein
Z¹ is selected from the group consisting of CH and N;
A is a ring selected from the group consisting of 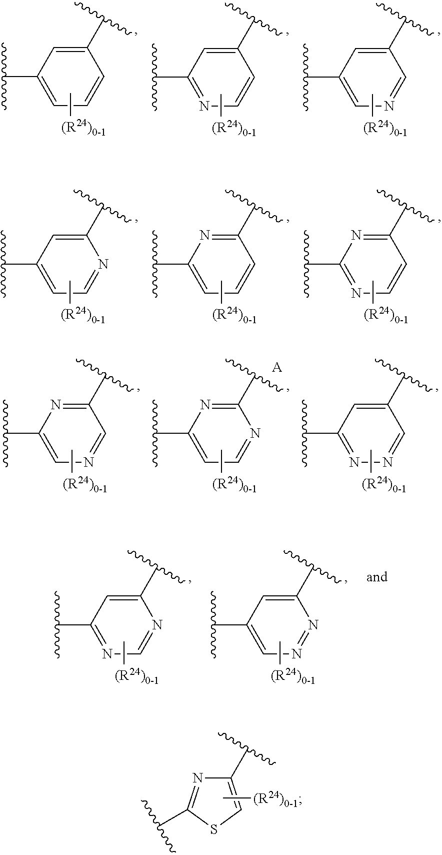
D¹ is CH, CR²⁴ or N;
R²⁷ is selected from the group consisting of
1) hydrogen,
2) C₁₋₆ alkyl wherein the alkyl group may be unsubstituted or substituted with 1-3 fluorine atoms and unsubstituted or monosubstituted with OC₁₋₃ alkyl,
3) C₃₋₆ cycloalkyl wherein the cycloalkyl group may be unsubstituted or substituted with 1-3 fluorine atoms and unsubstituted or monosubstituted with OC₁₋₃ alkyl, and
4) phenyl, wherein the phenyl group is unsubstituted or substituted with C₁₋₄ alkyl, —OC₁₋₄ alkyl, halogen, CN, NO₂, and S(O)₀₋₂C₁₋₄ alkyl, wherein C₁₋₄ alkyl and —OC₁₋₄ alkyl are unsubstituted or substituted with 1-3 flourine atoms;
L¹ is selected from the group consisting of O, S, C(R³²)2; and CF₂;
L² is selected from the group consisting of (CH₂)₂₋₄, —C(R³²)2, —CF₂—O, and S, provided that when L¹ is O or S, L² is not O or S;
R³² is independently selected from the group consisting of hydrogen and C₁₋₃ alkyl, wherein C₁₋₃ alkyl is unsubstituted or substituted with 1-3 flourine atoms;
E is a ring selected from the group consisting of
1) a 6-10 membered aryl ring
2) a 5-10 membered heteroaryl ring having 1, 2 or 3 heteroatoms independently selected from the group consisting of 0, 1, 2 and 3 N atoms, 0 or 1 O atoms, and 0 or 1 S atoms,
3) a C₃₋₈ cycloalkyl ring; wherein aryl, heteroaryl, and C₃₋₈ cycloalkyl are unsubstituted or monosubstituted with R²⁵, and unsubstituted or monosubstituted with R²⁵, and unsubstituted, monosubstituted or independently disubstituted with R²⁸,
R²⁴ in each instance in which it occurs, is independently selected from the group consisting of halogen,
C₁₋₆ alkyl, wherein the alkyl group may be unsubstituted or substituted with 1-3 fluorine atoms,
—O—C₁₋₆ alkyl, wherein the alkyl group may be unsubstituted or substituted with 1-3 fluorine atoms,
C₃₋₈ cycloalkyl, unsubstituted or substituted with 1-3 fluorine atoms, CN, and
NO₂;
R²⁵, in each instance in which it occurs, is independently selected from the group consisting of
1) R²⁶,
2) —OR²⁶,
3) C₁₋₆ alkyl which may be unsubstituted or substituted with 1-3 fluorine atoms, and unsubstituted or monosubstituted with a group independently selected from C₃₋₆ cycloalkyl, —O—C₁₋₄ alkyl, OH, ═O, S(O)₀₋₂C₁₋₄ alkyl, —OR²⁶ and R²⁶,
4) C₁₋₆ alkenyl which may be unsubstituted or substituted with 1-3 fluorine atoms and unsubstituted or monosubstituted with a group independently selected from —O—C₁₋₄ alkyl, OH, —O, S(O)₀₋₂C₁₋₄ alkyl, —OR²⁶ and R²⁶,
5) O—C₁₋₆ alkyl wherein the alkyl group may be unsubstituted or substituted with 1-3 fluorine atoms, and unsubstituted or monosubstituted with a group independently selected from C₃₋₆ cycloalkyl and R²⁶,
6) —S—C₁₋₆ alkyl,
7) a C₃₋₈ cycloalkyl ring which is unsubstituted or mono, di- or trisubstituted with groups independently selected from fluoro and C₁₋₄ alkyl, and unsubstituted or monosubstituted with a group independently selected from C₁₋₄ alkyl wherein the alkyl group may be unsubstituted or substituted with 1-3 fluorine atoms, —O—C₁₋₄ alkyl, OH, ═O, S(O)₀₋₂C₁₋₄ alkyl, —OR²⁶, R²⁶, and NR²⁹R³⁰,
8) a C₅₋₈ cycloalkenyl ring which is unsubstituted or mono, di- or trisubstituted with a group independently selected from fluoro and C₁₋₄ alkyl, and unsubstituted or monosubstituted with a group independently selected from C₁₋₄ alkyl, wherein the alkyl group may be unsubstituted or substituted with 1-3 fluorine atoms, —O—C₁₋₄ alkyl, OH, ═O, S(O)₀₋₂C₁₋₄ alkyl, and R²⁶,
9) a 5- to 6 membered heterocyclyl ring having 1 or 2 heteroatoms selected from the group consisting of N, O and S, and which is unsubstituted or monosubstituted with a group independently selected from C₁₋₄ alkyl wherein the alkyl group may be unsubstituted or substituted with 1-3 fluorine atoms, —OC₁₋₄ alkyl and ═O, and
10)halogen;
R²⁶ is selected from the group consisting of
1) a phenyl ring which is unsubstituted, monosubstituted or disubstituted with a group independently selected from the group consisting of halogen, OH, CN, C₁₋₄ alkyl wherein the alkyl group may be unsubstituted or substituted with 1-3 fluorine atoms, OC₁₋₄ alkyl wherein the alkyl group may be unsubstituted or substituted with 1-3 fluorine atoms, NO₂, S(O)₀₋₂C₁₋₄ alkyl, C₂₋₄ alkenyl, O—C₂₋₄ alkenyl, NR²⁹R³⁰, and COOH, and
2) a 5-6 membered heteroaryl ring containing 1-2 heteroatoms which are independently selected from N, O and S, wherein the heteroaryl ring is unsubstituted, monosubstituted or disubstituted with a group independently selected from: halogen, OH, CN, C₁₋₄ alkyl wherein the alkyl group may be unsubstituted or substituted with 1-3 fluorine atoms, OC₁₋₄ alkyl wherein the alkyl group may be unsubstituted or substituted with 1-3 fluorine atoms, NO₂, S(O)₀₋₂C₁₋₆ alkyl, S(O)₀₋₂ aryl, C₂₋₆ alkenyl, OC₂₋₆ alkenyl, NR²⁹R³⁰, and COOH;
R²⁸ is selected from the group consisting of
C₁₋₄ alkyl wherein the alkyl group may be unsubstituted or substituted with 1-3 fluorine atoms,
C₂₋₄ alkenyl,
halogen,
C₃₋₆ cycloalkyl, wherein the cycloalkyl group may be unsubstituted or substituted with 1-3 fluorine atoms,
OC₁₋₄ alkyl wherein the alkyl group may be unsubstituted or substituted with 1-3 fluorine atoms,
O—C₂₋₄ alkenyl,
NO₂,
S(O)₀₋₂C₁₋₄ alkyl, and
CN;
R²⁹ and R³⁰ are independently selected from the group consisting of hydrogen and C₁₋₆alkyl;
and
R³¹ is selected from the group consisting of hydrogen and C₁₋₆ alkyl. or a pharmaceutically acceptable salt or hydrate thereof.

10. The method according to claim 1 wherein activator is a compound of general formula (IV): 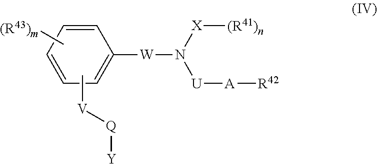
in which
V is absent, O, S, or NR⁴⁴, in which
R⁴⁴ is hydrogen or methyl,
Q is absent, straight-chain or branched alkylene having up to 9 carbon atoms or straight-chain or branched alkenediyl or straight-chain or branched alkinediyl having up to 4 carbon atoms which may be monosubstituted by halogen,
Y is H, NR⁴⁸R⁴⁹, cyclohexyl, phenyl, naphthyl or a heterocycle from the group consisting of 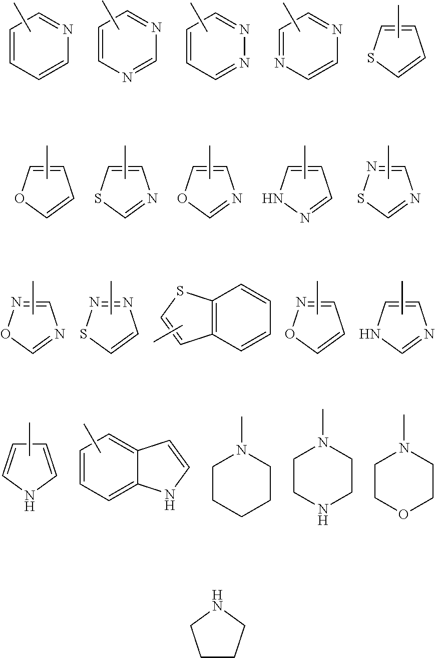
which may also be attached via N,
where the cyclic radicals may in each case be mono-, di or trisubstituted by straight-chain or branched alkyl, straight-chain or branched alkenyl, straight-chain or branched alkinyl, straight-chain or branched alkoxy, straight-chain or branched alkoxyalkoxy, straight-chain or branched halogenalkyl, straight-chain or branched halogenoalkoxy having in each case up to 4 carbon atoms, straight-chain or branched cycloalkyl having 3 to 6 carbon atoms, F, Cl, Br, I, NO₂, SR⁴⁶, NR⁴⁸R⁴⁹, NR⁴⁷COR⁵⁰ or CONR⁵¹R⁵²,
in which
R⁴⁶ is hydrogen, straight-chain or branched alkyl having up to 8 carbon atoms, or straight-chain or branched halogenoalkyl having up to 4 carbon atoms,
R⁴⁷ is hydrogen, or straight-chain or branched alkyl having up to 4 carbon atoms,
R⁴⁸, R⁴⁹, R⁵¹ and R⁵² independently of one another are hydrogen, straight-chain or branched alkyl having up to 4 carbon atoms or phenyl,
where the phenyl radical may be mono-, di- or trisubstituted by F, Cl, Br, hydroxyl, methyl, ethyl, n-propyl, i-propyl, n-butyl, s-butyl, i-butyl, t-butyl, methoxy, ethoxy, amino, acetylamino, NO₂, CF₃ OCF₃ or CN, or two substituents R⁴⁸ and R⁴⁹ or R⁵¹ and R⁵² may be attached to one another forming a five- or six-membered ring which may be interrupted by O or N,
R⁵⁰ is hydrogen, straight-chain or branched alkyl having up to 4 carbon atoms or phenyl,
where the phenyl radical may be mono- to trisubstituted by F, Cl, Br, hydroxyl, methyl, ethyl, n-propyl, i-propyl, n-butyl, s-butyl, i-butyl, t-butyl, methoxy, ethoxy, amino, acetylamino, NO₂, CF₃, OCF₃ or CN;
and/or the cyclic radicals may in each case be mono-, di- or trisubstituted by phenyl or a heterocycle from the group consisting of 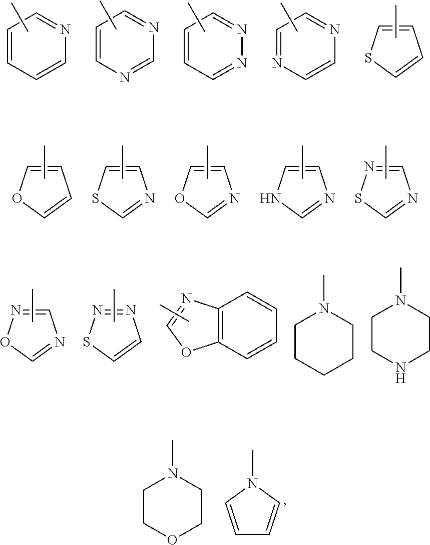
which may be attached directly or via a group O, S, SO, SO₂, NR⁴⁴, SO₂NR⁴⁷, CONR⁴⁷, straight-chain or branched alkylene, straight-chain or branched alkenediyl, straight-chain or branched alkyloxy, straight-chain or branched oxyalkyloxy, straight-chain or branched sulphonylalkyl, straight-chain or branched thioalkyl having in each case 4 carbon atoms and which may be mono- to trisubstituted by straight-chain or branched alkyl, straight-chain or branched alkoxy, straight-chain or branched alkoxyalkoxy, straight-chain or branched halogenoalkyl or straight-chain or branched alkenyl having in each case up to 4 carbon atoms, F, Cl, Br, I, CN, SCHL₃, OCF₃, NO₂, NR⁴⁸R⁴⁹ or NR⁵⁴COR⁵⁷,
in which
R⁵⁴ is hydrogen, straight-chain or branched alkyl having up to 8 carbon atoms, or cycloalkyl having 3 to 8 carbon atoms, and
R⁵⁷ is hydrogen, straight-chain or branched alkyl having up to 12 carbon atoms, straight-chain or branched alkenyl having up to 12 carbon atoms, aryl having 6 to 10 carbon atoms, an aromatic heterocycle having 1 to 9 carbon atoms and up to 3 heteroatoms from the group consisting of S, N and O or cycoalkyl having 3 to 8 carbon atoms, which may furthermore optionally be substituted by F, Cl Br, hydroxyl, methyl, ethyl, n-propyl, i-propyl, n-butyl, s-butyl, i-butyl, t-butyl, methoxy, ethoxy, amino, acetylamino, CO₂, CF₃, OCF₃ or CN;
and/or the cyclic radicals may be fused with an aromatic or saturated carbocycle having 1 to 10 carbon atoms or an aromatic or saturated heterocycle having 1 to 9 carbon atoms and up to 3 heteroatoms from the group consisting S, N and O,
R⁴³ is hydrogen or fluorine,
m is an integer from 1 to 2,
W is CH₂, —CH₂CH₂—, CH₂CH₂CH₂, CH═CHCH₂,
U is —CH₂—,
A is phenyl, pyridyl, thienyl or thiazolyl which may optionally be mono- to trisubstituted bymethyl, ethyl, n-propyl, i-propyl, n-butyl, i-butyl, s-butyl, t-butyl, CF₃, methoxy, ethoxy, F, Cl, Br,
R⁴² is COOR⁶⁴, in which
R⁶⁴ is hydrogen or straight-chain or branched alkyl having up to 4 carbon atoms,
X is straight-chain or branched alkylene having up to 8 carbon atoms or straight-chain or branched alkenediyl having up to 8 carbon atoms which may in each case contain one to three groups from the group consisting of phenyl, phenyloxy, O, CO and CONR⁷⁰, in which
R⁷⁰ is hydrogen, straight-chain or branched alkyl having up to 6 carbon atoms or cycloalkyl having 3 to 6 carbon atoms,
n is 1 or 2,
R⁴¹ is COOR⁷⁵, in which
R⁷⁵ is hydrogen or straight-chain or branched alkyl having up to 6 carbon atoms.

11. The method according to claim 10 wherein the activator is the compound of the following structure:

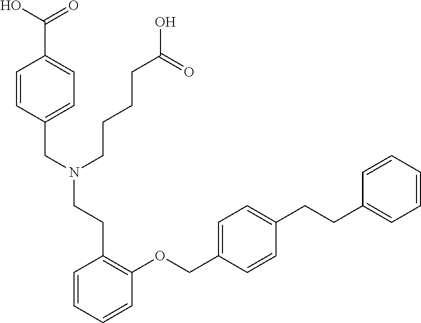 [SMILES: O=C(O)CCCCN(CCc1ccccc1OCc1ccc(CCc2ccccc2)cc1)Cc1ccc(C(=O)O)cc1]

12. The method according to claim 1 wherein the stimulator of the soluble guanylate cyclase or the activator of the soluble guanylate cyclase is adapted for systemic administration.

13. The method according to claim 1 wherein the stimulator of the soluble guanylate cyclase or the activator of the soluble guanylate cyclase is adapted for administration to a subject in single therapeutically effective daily doses thereof or multiple of therapeutically effective daily doses thereof.

14. A method according to claim 1 wherein the stimulator of the soluble guanylate cyclase is administered in a pharmaceutically effective dosage systemically.

15. The method according to claim 1 where the stimulator of the soluble guanylate cyclase or the activator of the soluble guanylate cyclase are employed in combination with a B-cell depleting agent.

16. The method according to claim 15 wherein the B-cell depleting agent is adapted for administration of one or two infusions twice within two weeks.

17. The method according to claim 15 wherein the B-cell depleting agent is a B-cell depleting anti-CD20 antibody or CD20 binding antibody fragment thereof, preferably, a monoclonal antibody or a CD20 binding antibody fragment thereof, like a humanized antibody or antibody fragment thereof or wherein the B-cell depleting agent is methotrexate.

18. The method according to any claim 15 wherein the stimulator of the soluble guanylate cyclase or the activator of the soluble guanylate cyclase, and the B-cell depleting agent are administered simultaneously, separately or sequentially to a subject suffering from chronic fatigue syndrome.

19-20. (canceled)

21. A composition containing a combination of a stimulator of the soluble guanylate cyclase and/or the activator of the soluble guanylate cyclase as defined in claim 1 and a B-Cell depleting agent.

22. The composition of claim 21 in form of a pharmaceutical composition for use in the treatment of chronic fatigue syndrome, in particular, for use in the treatment of chronic fatigue syndrome wherein the combination is administered simultaneously, separately or sequentially.
